(19) United States
(12) Patent Application Publication
(10) Pub. No.: US 2016/0058787 A1
(43) Pub. Date: Mar. 3, 2016

(54) Therapy and Cure of Ebola

(71) Applicant: Kumarpal A. Shah, Searingtown, NY (US)

(72) Inventor: Kumarpal A. Shah, Searingtown, NY (US)

(21) Appl. No.: 14/469,917

(22) Filed: Aug. 27, 2014

(51) Int. Cl. A61K 31/795; A01N 41/04

(52) U.S. Cl. CPC A61K 31/795; A01N 41/04

(57) ABSTRACT

A therapeutic model for the expedient treatment of deadly pathogens involved in pandemics and bioterrorism related events. The immune pathogenesis of deadly pathogens is redefined in the light of Recent advances in the Fundamentals of Immunology by developing three dimensional understandings of deadly pathogens and its interactions with host and its immune system. The immune pathogenesis of deadly pathogens can be treated expediently and globally with NSPS to mitigate the threat of bioterrorism and pandemics. According to a method for treating deadly pathogens having an immune regulatory molecule, the first step is providing a nano-engineered formulation of sodium polystyrene sulfonate (NSPS) having particle size less than 100 nm. A pharmaceutically effective dose of the NSPS is administered to a patient infected with the pathogen. The immune regulatory molecule is targeted with the NSPS for inhibiting serine protease activation. The therapeutic model is further extended for quarantine purposes to facilitate decontamination measures for patients, hospitals and laboratories.

### BACKGROUND OF THE INVENTION

1. Field of the Invention

This invention relates to the development of a model for the expedient therapy and cure of deadly pathogens globally. Ebola is used as a reference example.

More specifically, this invention exploits the recent advances in the Fundamentals of Immunology to develop a three-dimensional model of interactions of pathogens with host and its immune system. This model provides highly predictive tool to identify the earliest events in immune pathogenesis of deadly pathogens.

Based on above model, the invention details novel target molecules that can be targeted to inhibit immune pathogenesis safely without any adverse effects and toxicities to host. Novel Nano-formulation of an FDA approved, established and globally available drug, sodium polystyrene sulfonate as NSPS is detailed that targets safely the earliest event in the immune pathogenesis of deadly pathogens.

The invention also details formulation variations of NSPS that can be used as novel quarantine measures for the decontamination and eradication of deadly pathogens as well as to vaccinate vulnerable populations.

2. The Prior Art

There is an urgent global need to save lives in pandemics and Bioterrorism related attacks. Such needs are often measured in days and not in months and years. At stacks are lives of masses, productive work forces, global trade and freedom of the nations. Currently there are no adequate methods to treat and cure deadly pathogens such as Ebola. Also quarantine measures lack methods to sterilize asymptomatic contacts and its surrounding infectious environment.

The following website http://ebola.thelancet.com details current and evolving treatment and issues surrounding Ebola. The website is constantly updated for new advances.

Joint information Center, Emergency Operation Center, Center for Disease Control and Prevention on Aug. 13, 2014 published following details on Ebola that is subject to constant updates.

Key Points—Ebola Virus Disease, West Africa

This document is organized under following headings:
Summary Key Messages
Ebola Cases and Deaths (West Africa)
Ebola in U.S. Healthcare Workers (in Liberia)
Background on Ebola
Symptoms
Risk
Prevention
Treatment
What CDC is doing?
CDC Recommendations and Guidance
For More Information about Ebola

### Summary Key Messages

a. This is the largest Ebola outbreak in history and the first in West Africa.
b. The outbreak in West Africa is worsening, but CDC, along with other U.S. government agencies and international partners, is taking steps to respond to this rapidly changing situation.
c. Ebola poses no substantial risk to the U.S. general population.
d. On August 8, the World Health Organization (WHO) declared that the current Ebola outbreak is a Public Health Emergency of International Concern (PHEIC).
e. The PHEIC declaration underscores the need for a coordinated international response to contain the spread of Ebola.
f. Information about the PHEIC declaration is available on the WHO website www.who.int/mediacentre/news/statements/2014/ebola-20140808/en/#.
g. A person infected with Ebola virus is not contagious until symptoms appear.
h. The virus is spread through direct contact (through broken skin or mucous membranes) with the body fluids (blood, urine, feces, saliva, and other secretions) of a person who is sick with Ebola, or with objects like needles that have been contaminated with the virus, or infected animals.
i. Ebola is not spread through the air or by food or water.
j. As of August 13, no confirmed Ebola cases have been reported in the United States, other than the two U.S. health workers evacuated from Liberia.
k. As a precaution, CDC is communicating with American healthcare workers about how to isolate patients with known and suspected cases of Ebola and how they can protect themselves from infection.
l. Early recognition of Ebola is important for providing appropriate patient care and preventing the spread of infection. Healthcare providers should be alert for and evaluate any patients suspected of having Ebola.
m. A case definition for Ebola virus disease is available at www.cdc.gov/vhf/ebola/hcp/case-definition.html.
n. CDC and its partners at U.S. ports of entry are not doing enhanced screening of passengers traveling from the affected countries. However, CDC works with international public health organizations, other federal agencies, and the travel industry to identify sick travelers arriving in the United States and take public health actions to prevent the spread of communicable diseases.
o. CDC also is assisting with exit screening and communication efforts in West Africa to prevent sick travelers from getting on planes.
p. CDC recommends that people avoid nonessential travel to Guinea, Liberia, and Sierra Leone.
q. CDC recommends that people practice enhanced precautions if traveling to Nigeria.
r. Recommendations and guidance may change as new information becomes available.

### Ebola Cases and Deaths (West Africa)

As of Aug. 9, 2014, a total of 1848 suspected and confirmed cases of Ebola and 1013 deaths have been reported.

Guinea reported 506 cases, including 373 fatalities

Sierra Leone reported 730 cases, including 315 fatalities

Liberia reported 599 cases, including 323 fatalities

Nigeria reported 13 cases, including 2 fatalities

On July 25, the Nigerian Ministry of Health confirmed that a man in Lagos died from Ebola infection. The man had been in a hospital since arriving at the Lagos airport from Liberia. Additional Ebola cases have since been reported.

The death rate in some Ebola outbreaks can be as high as 90%, but in this outbreak it is currently around 55%-60%.

For specific areas where cases have been identified, see CDC's Ebola outbreak webpage.

### Ebola in U.S. Healthcare Workers (in Liberia)

Two U.S. citizens working at a hospital in Monrovia, Liberia, were confirmed to have Ebola virus infection in late July.

Both patients were safely transported to a hospital in the United States. A CDC Health Alert Network (HAN) notice describing the report of the two American healthcare workers and providing guidance to U.S. healthcare workers and hospitals regarding Ebola virus disease was distributed by CDC on July 28 (http://emergency.cdc.gov/han/han00363.asp).

### Background on Ebola

Ebola, also known as Ebola hemorrhagic fever, is a rare and deadly disease caused by infection with one of the Ebola virus strains (Zaire, Sudan, Bundibugyo, or Tai Forest virus).

Ebola viruses are found in several African countries. The first Ebola virus was discovered in 1976 near the Ebola River in what is now the Democratic Republic of the Congo. Since then, outbreaks have appeared sporadically in Africa.

The natural reservoir host of Ebola virus is not known. However, based on evidence and the nature of other similar viruses, researchers believe that Ebola virus is animal-borne, with bats probably being the most likely reservoir.

Ebola is spread through direct contact (through broken skin or mucous membranes) with a sick person's blood or body fluids, such as urine, saliva, feces, vomit, and semen. It is also spread through contact with contaminated objects (like syringes) or infected animals.

The incubation period, from exposure to when signs or symptoms appear, ranges from 2 to 21 days.

Genetic analysis of the virus indicates it is closely related to variants of Ebola virus (species Zaire Ebola virus) identified earlier in the Democratic Republic of the Congo and Gabon.

### Symptoms

The most common symptom of Ebola is fever.

Other symptoms include headache, joint and muscle aches, sore throat, and weakness, followed by diarrhea, vomiting, and stomach pain.

Other possible symptoms that might be seen in patients are skin rash, red eyes, and internal and external bleeding.

### Risk

Healthcare providers caring for Ebola patients and the family and friends in close contact with Ebola patients are at the highest risk of getting sick because they may come in contact with the blood or body fluids of sick patients.

People also can become sick with Ebola after coming in contact with infected wildlife. For example, hunting and preparing infected wildlife for bush meat.

### Prevention

There is no vaccine for Ebola.

If you must travel to an area affected by the Ebola outbreak, make sure to do the following:

Practice careful hygiene. Avoid contact with blood and body fluids.

Do not handle items that may have come in contact with an infected person's blood or body fluids.

Avoid funeral or burial rituals that require handling the body of someone who has died from Ebola.

Avoid contact with animals or raw meat.

Avoid hospitals where Ebola patients are being treated. The U.S. Embassy or consulate is often able to provide advice on facilities.

Seek medical care immediately if you develop fever, headache, muscle pain, diarrhea, vomiting, stomach pain, or unexplained bruising or bleeding.

Limit your contact with other people when you go to the doctor. Do not travel anywhere else.

After you return, pay attention to your health.

Monitor your health for 21 days if you were in an area with an Ebola outbreak, especially if you were in contact with blood or body fluids, items that have come in contact with blood or body fluids, animals or raw meat, or hospitals where Ebola patients are being treated or participated in burial rituals.

Seek medical care immediately if you develop fever, headache, muscle pain, diarrhea, vomiting, stomach pain, or unexplained bruising or bleeding.

Tell your doctor about your recent travel and your symptoms before you go to the office or emergency room. Advance notice will help your doctor care for you and protect other people who may be in the office.

### Treatment

There is no known, proven treatment for Ebola. Standard treatment for Ebola is limited to treating the symptoms as they appear and supportive care.

Experimental treatments have been tested and proven effective in animals but have not yet been tested in humans.

ZMapp, developed by Mapp Biopharmaceutical Inc., is an experimental treatment for use with persons infected with Ebola virus. The product is a combination of three different monoclonal antibodies that bind to the protein of Ebola virus.

It is too early to know whether ZMapp is effective or not because the drug is still in an experimental stage and has not yet been tested in humans for safety or effectiveness. Some patients infected with Ebola virus do get better spontaneously or with supportive care.

The best way to know if treatment with the product is effective is to conduct a randomized controlled clinical trial in people to compare outcomes of patients who receive the treatment to patients who have not. No such studies have been conducted to date.

### What CDC is Doing?

CDC has activated its Emergency Operations Center (EOC) to help coordinate technical assistance and control activities with partners.

On August 6, CDC elevated the EOC to Level 1 activation, its highest level, because of the significance of the outbreak.

CDC is in regular communication with other U.S. government agencies that are participating in the response, the ministries of health of the affected countries, the World Health Organization (WHO), and other international partners.

CDC has deployed several teams of public health experts to the West Africa region. More than 40 CDC staff deployed in Guinea, Sierra Leone, Liberia, and Nigeria are assisting with various response efforts, including surveillance, contact tracing, database management, and health education.

Over the next month, CDC plans to send additional public health experts to the affected countries to expand current response activities.

CDC staff are assisting with setting up an emergency response structure, contact tracing, providing advice on exit screening and infection control at major airports, and providing training and education in the affected countries.

CDC has issued a Warning, Level 3 notice for U.S. citizens to avoid nonessential travel to the West African nations of Guinea, Liberia, and Sierra Leone. CDC also has issued an Alert, Level 2 travel notice to advise about enhanced precautions for people traveling to Nigeria.

At this time, CDC is not doing enhanced screening of arriving travelers at U.S. airports, seaports, or land borders.

CDC is working closely with Customs and Border Protection (CBP) and other partners at ports of entry (primarily international airports) to use routine processes to identify travelers who show signs of infectious disease. In response to the outbreak, these processes have been enhanced through guidance and training. CDC's quarantine station staff is asked to respond as needed, for example by evaluating travelers identified by CBP officers.

If an ill traveler is identified during or after a flight, CDC will conduct an investigation of exposed travelers and work with the airline, federal partners, and state and local health departments to notify them and take any necessary public health action.

CDC is assisting with exit screening and communication efforts in West Africa to prevent sick travelers from getting on planes.

CDC has released interim guidance for airline flight crews, cleaning personnel, and cargo personnel that can be found at www.cdc.gov/quarantine/air/managing-sick-travelers/ebola-guidance-airlines.html

CDC has developed and posted Ebola-specific travel messages for electronic monitors to reach travelers from West Africa and posters for TSA screening areas of airports to reach outbound travelers. Visit wwwnc.cdc.gov/travel/page/info graphics-travelers to see the messages.

CDC is actively working to educate American healthcare workers on how to isolate patients and how to protect them from infection.

On August 1, CDC sent out a Health Alert Network (HAN) advisory (www.bt.cdc.gov/han/han00364.asp) informing U.S. healthcare workers of guidelines for evaluating U.S. patients suspected of having Ebola.

CDC has developed guidance for U.S. healthcare providers outlining how to prevent and control infections in hospitalized patients with known or suspected Ebola. This guidance can be found at www.cdc.gov/vhf/ebola/hcp/infection-prevention-and-control-recommendations.html.

On July 28, CDC sent out a HAN advisory (http://emergency.cdc.gov/han/han00363.asp) urging U.S. healthcare workers to be alert for signs and symptoms of Ebola in patients who have a recent travel history to countries where the outbreak is occurring.

CDC continues to update its communication products and webpages with new information on the Ebola outbreak for the general public and specific audiences.

A Questions and Answers on Ebola document (www.cdc.gov/vhf/ebola/outbreaks/guinea/qa.html) was posted on CDC's Ebola website and will be updated regularly.

CDC is working with partners to display Ebola-specific travel messages for electronic monitors and posters at ports of entry to reach travelers from West Africa.

CDC is using social media as a way to share credible, fact-based information and to dispel misconceptions about Ebola.

CDC hosted a Twitter chat about Ebola and the current outbreak on August 4. The chat was the largest chat in CDC history and provided the public an opportunity to have direct access to CDC's disease detectives. The potential reach of the chat was over 109 million.

### CDC Recommendations and Guidance

a. Healthcare Workers in West Africa

Healthcare workers who may be exposed to people with Ebola should follow these steps:

Wear protective clothing, including masks, gloves, gowns, and eye protection.

Practice proper infection control and sterilization measures. For more information, see “Infection Control for Viral Hemorrhagic Fevers in the African Health Care Setting” (www.cdc.gov/vhf/abroad/vhf-manual.html).

Isolate patients with Ebola from other patients.

Avoid direct contact with the bodies of people who have died from Ebola.

Notify health officials if you have been exposed to someone with Ebola.

b. Healthcare Providers in the United States

CDC encourages all U.S. healthcare providers to

Ask patients about their travel histories to determine if they have traveled to West Africa within the last three weeks.

Know the symptoms of Ebola—fever, headache, joint and muscle aches, weakness, diarrhea, vomiting, stomach pain and lack of appetite, and in some cases bleeding.

Know what to do if they have a patient with Ebola symptoms:
a. First, properly isolate the patient.
b. Then, follow infection control precautions to prevent the spread of Ebola.
c. Avoid contact with blood and body fluids of infected people.

U.S. healthcare workers should follow CDC's “Infection Prevention and Control Recommendations for Hospitalized Patients with Known or Suspected Ebola Hemorrhagic Fever in U.S. Hospitals” (www.cdc.gov/vhf/ebola/hcp/infection-prevention-and-control-recommendations.html).

CDC recommends standard, contact, and droplet precautions for management of hospitalized patients with known or suspected Ebola. These precautions can be found in “2007 Guideline for isolation Precautions: Preventing Transmission of Infectious Agents in Healthcare Setting” at www.cdc.gov/hicpac/2007IP/2007ip_part3.html.

### Infection Control

Any U.S. hospital that is following CDC's infection control recommendations and that can isolate a patient in his or her own room is capable of safely managing a patient with Ebola virus disease.

These patients need intensive supportive care; any hospital that has this capability can safely manage these patients.

Standard, contact, and droplet precautions are recommended.

Early Recognition

Early recognition is critical for infection control. Any patient with a suspected case of Ebola needs to be isolated until diagnosis is confirmed or Ebola is ruled out.

Healthcare providers should consider travel history, symptoms, and risks of exposure before recommending Ebola diagnosis.

Patient Placement

Patients should be placed in a single patient room (containing a private bathroom) with the door closed.

Facilities should maintain a log of all persons entering the patient's room

Protecting Healthcare Providers

All persons entering the patient room should wear at least: gloves, gown (fluid resistant or impermeable), eye protection (goggles or face shield), and a facemask

Additional personal protective equipment (PPE) might be required in certain situations (e.g., copious amounts of blood, other body fluids, vomit, or feces present in the environment), including but not limited to double gloving, disposable shoe covers, and leg coverings

Healthcare providers should frequently perform hand hygiene before and after all patient contact, contact with potentially infectious material, and before putting on and upon removal of PPE, including gloves.

### Patient Care Equipment.

Dedicated medical equipment (preferably disposable, when possible) should be used for the provision of patient care.

All non-dedicated, non-disposable medical equipment used for patient care should be cleaned and disinfected according to the manufacturer's instructions and hospital policies.

### Patient Care Considerations

Limit the use of needles and other sharps as much as possible.

Phlebotomy, procedures, and laboratory testing should be limited to the minimum necessary for essential diagnostic evaluation and medical care.

All needles and sharps should be handled with extreme care and disposed in puncture-proof, sealed containers

Avoid aerosol-generating procedures. If performing aerosol-generating procedures, use a combination of measures to reduce exposures from patients with Ebola hemorrhagic fever. (See CDC's guidance for more details on how to perform aerosol-generating procedures safely: www.cdc.gov/vhf/ebola/hcp/infection-prevention-and-control-recommendations.html.)

### Environmental Infection Control

Diligent environmental cleaning and disinfection and safe handling of potentially contaminated materials is paramount, as blood, sweat, emesis, feces, and other body secretions represent potentially infectious materials.

Healthcare providers performing environmental cleaning and disinfection should wear recommended PPE (described above) and consider use of additional barriers (e.g., shoe and leg coverings) if needed.

Face protection (face shield or mask with goggles) should be worn when performing tasks such as liquid waste disposal that can generate splashes.

Follow standard procedures, per hospital policy and manufacturers' instructions, for cleaning and/or disinfection of:

Environmental surfaces and equipment

Textiles and laundry

Food utensils and dishware

### Duration of Precautions

The duration of precautions should be determined on a case-by-case basis, in conjunction with local, state, and federal health authorities.

For more details on infection control in U.S. hospitals, see www.cdc.gov/vhf/ebola/hcp/infection-prevention-and-control-recommendations.html.

### Travelers

CDC has issued a Warning, Level 3 travel notice for 3 countries, U.S. citizens should avoid all nonessential travel to Guinea, Liberia, and Sierra Leone.

CDC has issued an Alert, Level 2 travel notice for Nigeria. Travelers to Nigeria should take enhanced precautions to prevent Ebola.

If you travel to any of the four affected countries, make sure to do the following:

Practice careful hygiene. Avoid contact with blood and body fluids.

Do not handle items that may have come in contact with an infected person's blood or body fluids,

Avoid funeral or burial rituals that require handling the body of someone who has died from Ebola.

Avoid contact with animals or raw meat.

Avoid hospitals where patients with Ebola are being treated. The U.S. Embassy or consulate is often able to provide advice on facilities.

Seek medical care immediately if you develop fever, headache, muscle aches, diarrhea, vomiting, stomach pain, or unexplained bruising or bleeding.

Limit your contact with other people when you go to the doctor. Do not travel anywhere else.

Pay attention to your health after you return.

Monitor your health for 21 days if you were in an area with an Ebola outbreak, especially if you were in contact with blood or body fluids, items that have come in contact with blood or body fluids, animals or raw meat, or hospitals where patients with Ebola are being treated.

Seek medical care immediately if you develop fever, headache, muscle pain, diarrhea, vomiting, stomach pain, or unexplained bruising or bleeding.

Tell your doctor about your recent travel and your symptoms before you go to the office or emergency room. Advance notice will help your doctor care for you and protect other people who may be in the office.

Visit the CDC Travelers' Health website (www.cdc.gov/travel/) for more information about the outbreak and for other health recommendations to the specific countries.

Airline Flight crew's, Cleaning Personnel, and Cargo Personnel

CDC's Interim Guidance about Ebola Virus Infection for Airline Flight Crews, Cleaning Personnel, and Cargo Personnel is posted on CDC's website at www.cdc.gov/quarantine/air/managing-sick-travelers/ebola-guidance-airlines.html.

Monitoring and Movement of People with Ebola

DC has developed interim guidance to provide public health authorities and other partners a framework for evaluating people's risk of exposure to Ebola and initiating appropriate public health actions on the basis of exposure risk and clinical assessment.

The interim guidance describes public health actions for people with high risk, low risk, and no known exposure to Ebola. The guidance is available on CDC's website www.cdc.gov/vhf/ebola/hcp/monitoring-and-movement-of-persons-with-exposure.html.

### Laboratory Specimen Collection, Transport, and Testing

CDC has developed interim guidance for laboratories and other healthcare personnel who collect or handle specimens in the United States on the appropriate steps for collecting, transporting, and testing specimens from patients who are suspected to be infected with Ebola virus. The guidance is available on CDC's website www.cdc.gov/vhf/ebola/hcp/interim-guidance-specimen-collection-submission-patients-suspected-infection-ebola.html

For More Information about Ebola

CDC will continue to post new information about the Ebola outbreak on the following websites as it becomes available:

CDC Ebola Hemorrhagic Fever site: www.cdc.gov/ebola

CDC Travelers' Health site: http://wwwnc.cdc.gov/travel/notices

World Health Organization (WHO) Ebola virus disease (EVD) site: www.who.int/csr/disease/ebola/en/.

Ebola safety data sheet to highlight the dangerous nature of pathogen and the need for extreme precautions. These are summarized at http://www.phac-aspc.gc.ca/lab-bio/res/psds-ftss/ebola-eng.php Copyright© Public Health Agency of Canada, 2010.

Rosenthal A “Controlling the Ebola Epidemic” The New York Times editorial, A16, Aug. 11, 2014 (1) summarizes the current problems in Ebola and the urgency to stem out its outbreak.

### Statement of the Problem:

On Friday, Aug. 8, 2014, World Health Organization formally declared an international public health emergency in response to what its director general, Dr. Margaret Chan, called “the largest, most severe, most complex outbreak” of the deadly Ebola virus “in the nearly four decade-decade history of the disease.” And what the World has done in those last forty years to defend against the disease? Not much. Apart from inflicting a staggering human toll, the spread of the Ebola virus in West Africa has laid bare how unprepared the United States and other advanced countries are to protect and treat thousands of Africans whose lives are threatened by an extremely dangerous virus for which there is no cure. As the Centers for Disease Control and Prevention puts it: “We do not know how to treat Ebola or vaccinate against it—and it will be a long time before we do.”

ii. Statistics as of Aug. 8, 2014: The W.H.O. reported that Ebola has infected about 1,800 people in four West African countries and killed almost 1,000 of them.

iii. The Problems in drug and vaccine treatment: The drugs that could potentially treat those already infected and the vaccines to protect healthy people from infection are all in the earliest stages of testing. And even if they do pass muster in clinical trials, they cannot be produced in large quantities quickly enough to stem the widening epidemic anytime soon.

These uncertainties were highlighted by the special treatment given to an American doctor and an American aid worker who were infected while caring for patients in Liberia and treated there with a drug that has worked well in monkeys but never been tested in humans. They were flown back to Atlanta to receive the best care possible in an isolation ward at Emory University Hospital. There were exuberant media reports that they had been saved by the drug and demands from Liberia that the drug be made available to African patients. Yet at this point no one knows whether the drug played any role in helping the two Americans, only that it didn't kill them.

In any case, very little of the drug is available, and the small company that makes it does not have the capacity to produce large quantities to treat large numbers of patients in the field. Another drug candidate is in clinical trials with a small number of patients, but the trials were recently suspended and then partially reinstated because of fears that it could harm the patients. Meanwhile, a clinical trial of the first potential vaccine is being expedited, but it will be months before its safety can be verified and the vaccine made available for humans.

As the Centers for Disease Control and Prevention puts it: “We do not know how to treat Ebola or vaccinate against it—and it will be a long time before we do.”

Many drug companies have little interest in devising treatments or vaccines for Ebola because the potential for profit is small. Much of the research has been financed by the National Institutes of Health and the Department of Defense and carried out by small start-ups, but some experts believe the federal government has not shown enough urgency to push these programs ahead.

Traditional public health measures, like finding and isolating patients who become sick, tracing their contacts and using stringent infection control procedures in hospitals, remain the best bet for containing the epidemic in West Africa.

The C.D.C. has elevated its response to the highest possible level and is sending 50 more health care professionals to the area, backed by hundreds more professionals in this country. Sierra Leone, which has the highest number of cases, is planning to deploy hundreds of troops and police officers to enforce isolation measures that its residents have so far ignored, and Liberia, with the second largest number, has declared a 90-day state of emergency that allows it to suspend civil liberties and impose quarantines. Nigeria has also declared a state of emergency. Such public health measures should ultimately, although perhaps not quickly, bring the outbreaks under control.

Current understanding of the 2014 Ebola is summarized in depth by Derek Gatherer in “The 2014 Ebola virus disease outbreak in West Africa” published in Journal of General Virology (2014), 95, 1619-1624 (2).

The current understandings of immune pathogenesis of Ebola are summarized as under. A. Ebola virus is resistant to immune attack. The underlying cause of immune resistance relates to immune evasion mechanism of Ebola. The mechanism of Ebola entry and immune evasion is summarized by
Ramanan P et al in “Filo viral Immune Evasion Mechanisms” published in Viruses 2011, 3, 1634-1649 Sep. 7, 2011 (3)
White J. M. and Schonberg K. L. in “A new player in the puzzle of filo virus entry” published in Nat Rev Microbial. 10 (5): 317-322. January 2013 (4); and
Osvaldo M. L. et al in “The role of antigen-presenting cells in filo viral hemorrhagic Fever: gaps in current knowledge” published in Antiviral Res. 2012 March; 93 (3): 416-428. Nov. 1, 2012 (5)

B. Ebola activates serine proteases to stimulate host inflammatory and bleeding responses. Lethal Ebola viral infections produce widespread inflammation with vascular leak, clotting, and bleeding (disseminated intravascular coagulation [DIC], organ failure, and high mortality. Serine proteases contribute to clot-forming (thrombotic) and clot-dissolving (thrombolytic) effects of DIC. The current knowledge of immune pathogenesis of Ebola involves interactions between the Coagulation and Complement System. Amara U. I. et al in Adv. Exp Med Biol. 2008; 632: 71-79, details this aspect (6).

Serine protease inhibitor was assessed in wild type B ALB/c mice adapted for Zaire Ebola Virus. The improved survival and reduced tissue necrosis was reported by Chen H et al in “Myxomavirus-Derived Serpin Prolongs Survival and Reduces Inflammation and Hemorrhage in an Unrelated Lethal Mouse Viral Infection”. The findings were published in Antimicrobial Agents and Chemotherapy p. 4114-4127 September 2013 Volume 57 Number 9 (7),

Current vaccine efforts are summarized in following articles.
Zhang APP et at in “The Ebola virus VP24 interferon antagonist Know your enemy” published in Virulence 3:5, 440-445; Aug. 15, 2012; G 2012 Landes Bioscience (8)
Vaccine efforts in Plant-produced Ebola Immune Complex as an Ebola Vaccine Candidate is detailed By Waranyoo Phooleharoen published in December 2010 Arizona State University (9).
Stoiber H in EP 2 208 737 A1 European patent application filed on Feb. 5, 2008 (10) details complement activating construct comprising a complement factor H-derived short consensus repeat (fh-derived SCR) and a binding molecule such as monoclonal Ab (SCR-AB Construct) which specifically recognizes pathogen as a method for the prevention, treatment and amelioration of infection. Ebola is one of the various viral pathogens listed for such therapy. The SCR-AB construct specifically recognizes the viruses that are double stranded DNA, single stranded DNA virus, double stranded RNA virus, positive-sense single stranded RNA, and negative-sense single stranded RNA virus or reverse transcribing RNA viruses. Ebola is a negative sense single stranded virus of family Filoviridae.

Following are the additional examples of viruses covered in the patent applications.

### Viral Family:

Flaviviridae (Positive sense single stranded RNA virus) Examples are Hepatitis C and Yellow fever viruses)

Retroviridae (Reverse transcribing RNA virus) Example Human deficiency virus and Friend Murine Leukemia virus

Paramyxoviridae (Negative sense single strand RNA) examples are Measles virus

Rhabdoviridae (Negative sense single-stranded RNA virus, Example are rabies virus)

Herpesviridae (double stranded DNA Virus) Example is Epstein-Barr virus

Hepadnasvirides (Reverse transcribing DNA Virus) Example Hepatitis B virus

The underlying hypothesis is deadly pathogens are resistant to complement mediated lysis due to the presence of immune regulatory molecules. Complement activating-SCR construct when combined with specific monoclonal Abs, it will and removal of such immune regulatory molecules to prime Cytotoxic immune responses.

Above therapeutic efforts are at early stage of investigations. According to another editorial in New York Times on Aug. 24, 2014 detailed at http://www.nytimes.com/2014/08/25/opinion/new-thinking-about-ebola-treatments.html?_r=1 there is a growing consensus amongst experts to use radical approaches to use established drug and reinvent its applications for Ebola Therapy. This approach however, is associated with many dangers and unanswered questions.

### SUMMARY OF THE INVENTION

It is the first object of the invention to identify the earliest event that contributes to the highly infectious nature of deadly pathogens such as Ebola.

It is the second object of the invention to identify the earliest event in the immune pathogenesis of Ebola that is responsible for the current deadly symptoms and signs of Ebola such as hemorrhagic fever and host inflammatory responses.

It is the third object of the invention to identify the earliest immune evasion mechanism, which allows Ebola to escape host immune surveillance, detection and attack mechanism against pathogens. Instead of protecting host, the host immune system allows deadly pathogens such as Ebola to survive and thrive inside host immune cells. This leads to the destruction of host immune system and mount vicious inflammatory and hemorrhagic attacks on host tissues leading to the demise of host and the spread of infections through contamination of host surroundings.

These and related objects are achieved by providing a therapeutic model based on the three dimensional interactions of deadly pathogens with host and its immune system. This model is based on the recent advances in the fundamentals of immunology and provides the expedient treatment of deadly pathogens involved in pandemics and bioterrorism related events. The immune pathogenesis of deadly pathogens can be treated expediently and globally with nano-engineered formulation of sodium polystyrene sulfonate NSPS to mitigate the threat of bioterrorism and pandemics.

These objects are achieved by targeting one or more of Serine proteases of coagulation cascade, classical complement system cascade, Lectin based complement cascade, Alternate complement based cascade and combinations thereof. The NSPS is used to down regulate host inflammatory, bleeding and cytokine inflammatory response. NSPS is used to target immune evasion mechanism in deadly pathogens. Targeting includes generating cytotoxic immune vaccine responses by inhibiting immune regulatory molecules located on chromosome 1 at Q32 position. In one example, the deadly pathogen is Ebola.

It is the fourth object of the invention to identify a drug that targets the earliest event that contributes to the highly infectious nature of deadly pathogens such as Ebola.

It is the fifth object of the invention to target the earliest event in the immune pathogenesis of Ebola.

It is the sixth object of the invention to identify a drug that targets the earliest immune evasion mechanism of Ebola.

It is the seventh object of the invention to identify an FDA approved drug that is established and globally available such as sodium polystyrene sulfonate (SPS) that has validated target data for items 000154-000165.

It is the eighth object of the invention to analyze safety and efficacy parameters of above drug SPS and improve its formulation methods as nano-polystyrene sulfonate (NSPS) to enhance the safety and efficacy by over 100 folds while overcoming prior art problems of pharmacokinetic limitations.

These and related objects are achieve by an embodiment of the invention presented as a method for treating deadly pathogens having an immune regulatory molecule. In the first step, a nano-engineered formulation of sodium polystyrene sulfonate (NSPS) is provided having particle size less than 100 nm. A pharmaceutically effective dose of the NSPS is administered to a patient infected with the pathogen. The immune regulatory molecule is targeted with the NSPS for inhibiting serine protease activation.

The method further comprises a therapeutic model for the expedient treatment of deadly pathogens involved in pandemics and bioterrorism related events, for example where the deadly pathogen is ebola. The immune pathogenesis of deadly pathogens is redefined in the light of Recent advances in the Fundamentals of Immunology. The immune pathogenesis of deadly pathogens can be treated expediently and globally with NSPS to mitigate the threat of bioterrorism and pandemics.

The providing step includes providing NSPS or providing milligram dosages of NSPS. The targeting step includes targeting one or more of the group consisting of Serine proteases of coagulation cascade, classical complement system cascade, Lectin based complement cascade, Alternate complement based cascade and combinations thereof. The targeting step includes using NSPS to down regulate host inflammatory, bleeding and cytokine inflammatory response. The targeting step includes using NSPS to target immune evasion mechanism in deadly pathogens. The targeting step includes generating cytotoxic immune vaccine responses by inhibiting immune regulatory molecules located on chromosome 1 at Q32 position.

The administering step includes administering an oral single daily dose of 5 mg NSPS per kg of the patient's body weight. Alternatively, administering by injection a 5 mg NSPS per kg of the patient's body weight suspended in one of sterile water or normal saline. A further route is administering by nasal spray 10-100 mg NSPS suspended in normal saline.

It is the ninth object of the invention to detail NSPS in its formulation variations for use in quarantine measures to internally sterilize and decontaminate patients and animal sources that harbor infectious pathogens and act as sources of future transmissions of deadly pathogens.

These objects are achieved in modifying the providing step to include centrifuging patient matched blood and collecting cells; combining 10-100 mg NSPS with the collected cells and incubating; and adding sterile solution to form a mixture. The mixture is then administered intravenously. The targeting step includes linking the NSPS to one or more of RBC, platelets, monocytes and white blood cells for removing interfering immune regulatory molecules and priming cytotoxic immune responses.

In an alternate approach, the providing step includes centrifuging patient matched blood and collecting cells; combining 10-100 mg NSPS with the collected cells and incubating; and adding sterile solution to form a mixture. The mixture is again administered intravenously. The targeting step includes linking the NSPS to suitable monoclonal antibodies for generating cytotoxic immune responses.

In a further variation of approach, the targeting step includes linking the NSPS with Ebola antigen for generating vaccine responses in healthy patients.

It is the tenth object of the invention to detail NSPS and its formulation variations for the external sterilization and decontamination of infectious area and work environment to prevent spread of diseases in healthcare workers and patient contacts.

These objects are achieved by modifying the NSPS formulation as a spray to bind pathogens and render it inactive in externally contaminated body fluid and its environments, garments, work areas including laboratory equipment and office equipment including smart phone, IPAD and files.

It is the eleventh object of the invention to apply above model of drug treatment, prophylaxis and quarantine measures to all lethal pathogens.

I achieve above objects of the inventions by:

Firstly, realizing that historically, fundamentals of immunology have contributed greatly to the most cost-effective solutions to eradicate deadly pathogens such as Smallpox and Polio.

Secondly, I have identified the key recent advances in the fundamentals of immunology. Three-dimensional interactions of deadly pathogens with host and its immune system contribute to the novel understandings of the earliest events in the immune pathogenesis of deadly pathogens.

Thirdly, I have screened for FDA approved drug list and literature database to identify a drug that is established and globally available (11). Top notched scientists have worked on this drug and have produced relevant scientific data for different purposes. They have reported their findings in peer reviewed scientific publications. A careful summation of above data covers key items and provides evidence based data for from first to eleventh objects of inventions (12-16).

Fourthly, I have identified the toxicities and pharmacokinetic limitations of FDA approved drug SPS and have used it first hand to develop nano formulation method to overcome prior art problems and improve the safety and efficacy of the drug.

I have used the nano formulated drug first hand to ascertain that it will not precipitate deadly host inflammatory responses while targeting immune pathogenesis in item First to Eleventh modes of invention as per recent advances in the fundamentals of immunology.

These aspects of inventions are detailed in stepwise manner in the following along with the diagrams and key references.

### BRIEF DESCRIPTION OF THE DRAWINGS

The advantages, nature, and various additional features of the invention will appear more fully upon consideration of the illustrative embodiments now to be described in detail in connection with accompanying drawings. In the drawings wherein like reference numerals denote similar components throughout the views.

FIG. 1 is a diagram showing Immune regulatory molecules on an Ebola surface.

FIG. 2 is a diagram showing the importance of Factor H in protecting Ebola.

FIG. 3 is a diagram showing the genomic location of Factor H to chromosome 1 at Q32 position.

FIG. 4A is an enlarged view showing the structure of Ebola.

FIG. 4B is an enlarged view showing the structure of Factor H.

FIG. 5 is a diagram showing the efficient virolysis by Targeting Factor H with Factor H Ab or NSPS.

FIG. 6 is a diagram showing the generation of Host specific immune responses.

### DETAILED DESCRIPTION OF THE PREFERRED EMBODIMENTS

The description below can identify the earliest event that contributes to the highly infectious nature of deadly pathogens such as Ebola.

Further it is the earliest event in the immune pathogenesis of Ebola that is responsible for the current deadly symptoms and signs of Ebola such as hemorrhagic fever and host inflammatory responses.

In addition this application identifies the earliest immune evasion mechanism, which allows Ebola to escape host immune surveillance, detection and attack mechanism against pathogens.

High degree of electrostatic interactions of deadly pathogens with host immune system subverts the protective function of host immune system on host cells. Instead of protecting host, the host immune system acts as a foe allowing deadly pathogens such as Ebola to survive and thrive inside host immune cells. This leads to the destruction of host immune system and mount vicious inflammatory and hemorrhagic attacks on host tissues leading to the demise of host and the spread of infections through contamination of host surroundings.

a. Historical Background: Genesis of above concepts resides in Greek mythology tells of Narcissus, a young man punished by the gods for scorning the many women who fell in love with him. Corning across a pool of clear water, Narcissus saw his own reflection. Made to believe that a spirit lived in the pool, he admired the beauty of the face reflected back at him; unwittingly, Narcissus fell in love with himself and died in the futility of his obsession, Narcissus's downfall wrought by his inability to identify the reflection as his own can be viewed as a metaphor for the fundamentals of immunology or concept of Self and Non-self (SNS).

Fundamentals of Immunology or the science of human defense or SNS concept is the “Heart of Medicine”. It is the central software that is relentlessly exploited by deadly pathogens causing global mortality in modern times. Historically, these advances have cured and eradicated deadly pathogens such as Smallpox and Polio. Inspite of the global efforts and technology advances, why then modern efforts fail to generate proper therapies and vaccines for deadly pathogens?

b. Our Efforts: Since 1980s we were studying and searching fundamentals of immunology to identify key recent advances in the fundamentals of immunology that can be used as a software to develop novel drugs and therapies for deadly pathogens. The success of our efforts is summarized in the answers to key series of questions that we identified as under.

Question 1: One of the key problems in the fundamentals of immunology is—Why normal host is protected when immune system is activated against pathogens?

Answer: Scientists explain this by stating that there are Immune regulatory Molecules present on host cell surface. They protect host when the immune system is activated against pathogens (Dierich M P, Stoiber H. and Clivio A. “Complement-ary AIDS Vaccine” Nature Medicine, 1996, 2: 153-155) (17).

Question 2: This is an opposite of above question. Why then deadly pathogens are protected by host immune system?

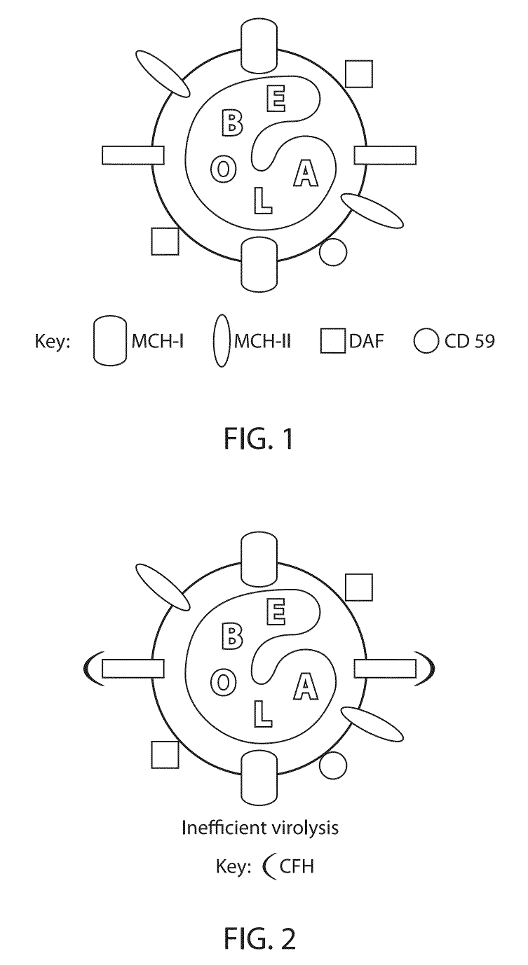

Answer: Scientists explain this by stating that the deadly pathogens acquire immune regulatory molecules of host when they bud out of host cell membrane after multiplying in host tissues. See FIG. 1

FIG. 1 is a diagram showing the presence of Immune regulatory molecules on an Ebola surface that has a coat of human cell membrane as per old or early or original concepts in Fundamentals of Immunology.

Recent Advances in Fundamentals of Immunology:

It has identified the dominant role of Factor H in host protection. According to this understanding it is the Factor H that protects the deadly pathogens such as Ebola from immune attack and prevent efficient virolysis (Walport M. J. “Advances in Immunology” Complement: First of two parts, N. Eng. J. of Med. 2001, 344:1058-1066) (18) and Joshua M. et al “The Central Role of the Alternative Complement Pathway in Human Disease” The Journal of Immunology, 2006, 176:1305-1310 (19).

FIG. 2 is a diagram showing the importance of Factor H in protecting Ebola as per new concept of Recent Advances in the Fundamentals of Immunology. In the diagram “(” represents Factor H also known as “CFH” Deadly pathogens bind to Factor H and simultaneously activate serine proteases. Both steps are necessary for viral protection from host immune system and to gain entry inside host immune cells. An appropriate sub-title for FIG. 2 would be Immune Pathogenesis of Deadly Viruses: The Role of Factor H as per recent advances in the Fundamentals of Immunology.

Based on above understandings Pinter C et al described “Interference with complement immune regulatory molecules as a possible therapeutic strategy in HIV infection” and published their work in Exp Opin. Invest Drug, 2000; 9:2; 197-205 (20).

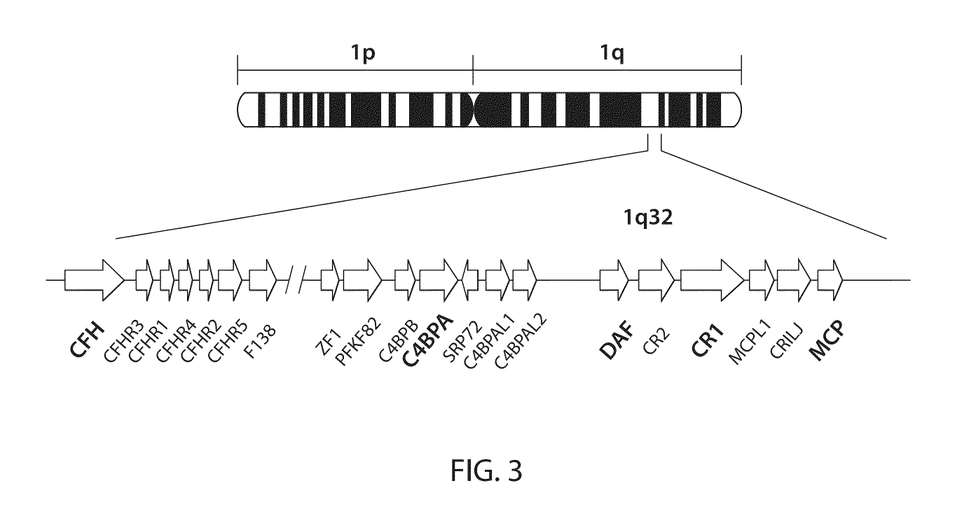

We have identified a therapeutic drug SPS to target immune regulatory molecule. Our communications and visit to Austrian group of investigators in early 1990s provided further insight (Letter Communication) (21). This is reflected in FIG. 3 through FIG. 6.

FIG. 3 is a diagram showing the genomic location of Factor H in chromosome 1 at Q32 position as per Recent Advances in the Fundamentals of Immunology. The recent advances in genomics details the genomic location of a molecule that protects host cells from viral invasion in normal subjects. It is a long-standing mystery how host protect against invading virus when immune system is activated. According to the recent advances in the fundamentals of immunology—this function is due to immune regulatory molecule is located in Chromosome 1 at 1Q32 position. It inactivates fluid serine protease immune activity normal host cells. When deadly pathogens such as Ebola invade—this is the first molecule Ebola binds and develops resistance to immune system.

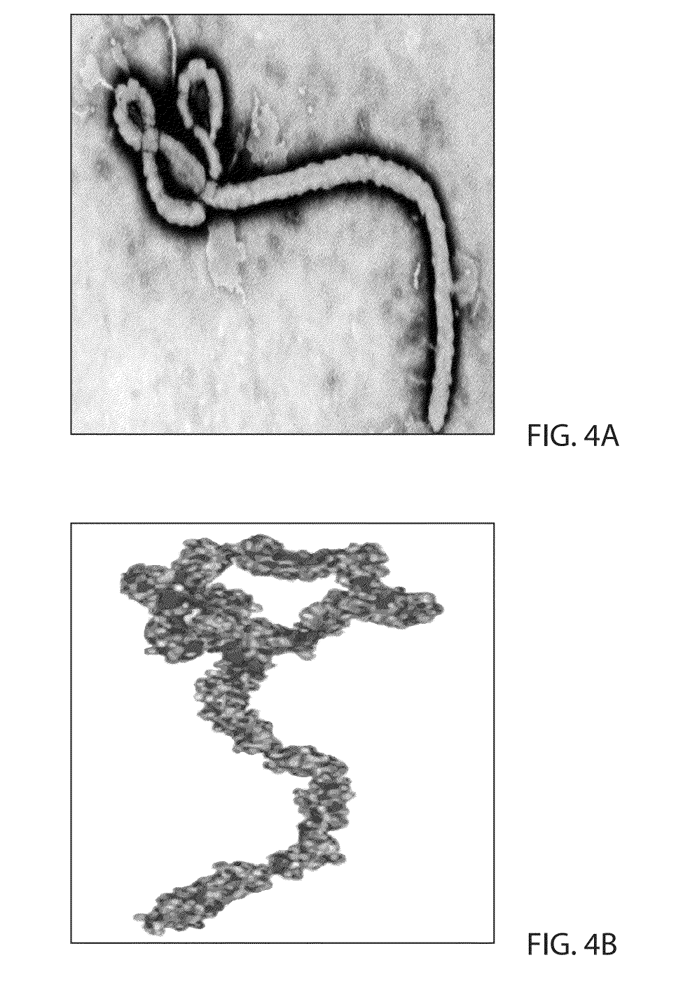

FIGS. 4A and 4B are provided to draw attention to highly electrostatic charges carried by Ebola as a representative example of deadly pathogen and Factor H as a representative example of Host immune regulatory molecule present in fluid as well as on host cell surface. High electrostatic charge present on both molecules contributes to binding with complement proteins either to inhibit its immune function (Factor H) or to activate its functions (Ebola). Ebola and Factor H have highly electrostatic properties contributing to their folded structure, as shown in FIGS. 4A and 4B, and its function of interactions with complement proteins. Ebola requires quarantine measures to protect eyes, hands, external mucous membrane of oral and genital surfaces, clothing and work environment.

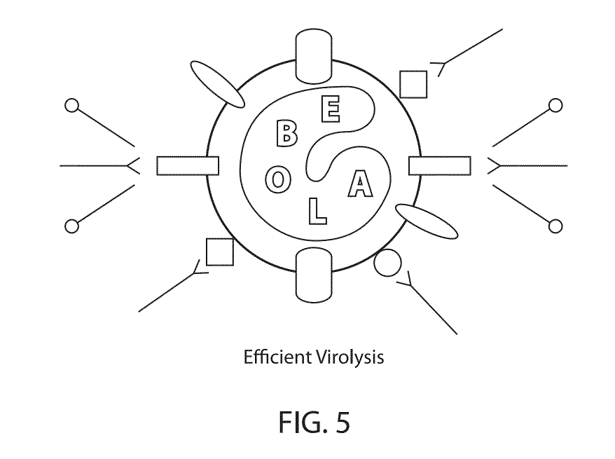

FIG. 5 shows Ebola masks its identity as “Self or Host cell” by binding with Factor H. However, it continues to activate Serine proteases (Ref. Example Factor D) to contribute to both host inflammations, bleeding and organ failures. FIG. 5 diagrams efficient virolysis by Targeting Factor H with Factor H Ab or NSPS. The advantage of NSPS is that it can also target the serine protease activation step preventing the proteolysis step necessary for viral entry in host cell. This concept can be extended for prophylaxis and quarantine measures.

Factor H has a normal function is to inhibit immune activation but when its function is subverted by deadly pathogen such as Ebola, mutated or if it is absent in blood due to rare abnormalities—it can precipitate severe Non-viral syndrome that is characterized by host inflammatory reactions, bleedings and organ failures. The non-viral syndrome is known as Atypical Hemolytic Uremia Syndrome (aHUS) Kavanagh D et al in “Atypical Hemolytic Uremia Syndrome” British Medical Bulletin, 2006, 77-78, 5-22 (22).

A careful perusal of the properties of our drug SPS revealed it is a broad-spectrum antiviral drug and targets serine proteases of coagulation and complement cascade. Additionally, it is a swellable decoy molecule like a living cell of 25-150 micron size that has affinity for potassium over sodium like a living cell. For this reason, in-vitro efficacy is not readily transferable to in-vivo efficacy (Mattll, Luscher M. Antiviral Chemistry & Chemotherapy. Polyanions: A lost chance in the fight against HIV and other virus diseases. International Medical Press, London. 2000 Jul. 11 (4):249-59) (23).

Our recent efforts were focused on improving the pharmacokinetic properties and improve safety and efficacy of the molecule. This we are able to achieve by developing Nano formulation of SPS as NSPS that has particle size of less than 100 nanometers. One critical question, we frequently encountered in our discussions with to most experts in immunology and infectious disease branches was that—ASPS may lead to AHUS like syndrome due to Factor H binding. Therefore we have carefully engineered the molecule to inhibit Factor H (immune regulatory molecule of Alternate complement system) at micro dose level and counter the immune activating effects of Factor D of Alternate complement system as a representative example of serine proteases at higher milligram dose level.

The success of this strategy is self-tested by using this drug in both micro gram doses in nasal formulations over two years and in milligram doses orally for over two years. The highlight of NSPS is that it reduces the toxicity and adverse effects of SPS by over 100 folds while overcoming the problems of prior art pharmacokinetic limitations. Formulation variations proposed are designed for therapy, cure, and vaccine responses safely and also for quarantine purposes:

1. For the Therapy of Ebola: Intravenous injection of NSPS at less than 5 mg/kg body weight. At this dose, it will inhibit serine proteases of coagulation and complement system to inhibit downstream pathways contributing to Host inflammatory cytokines and bleeding.

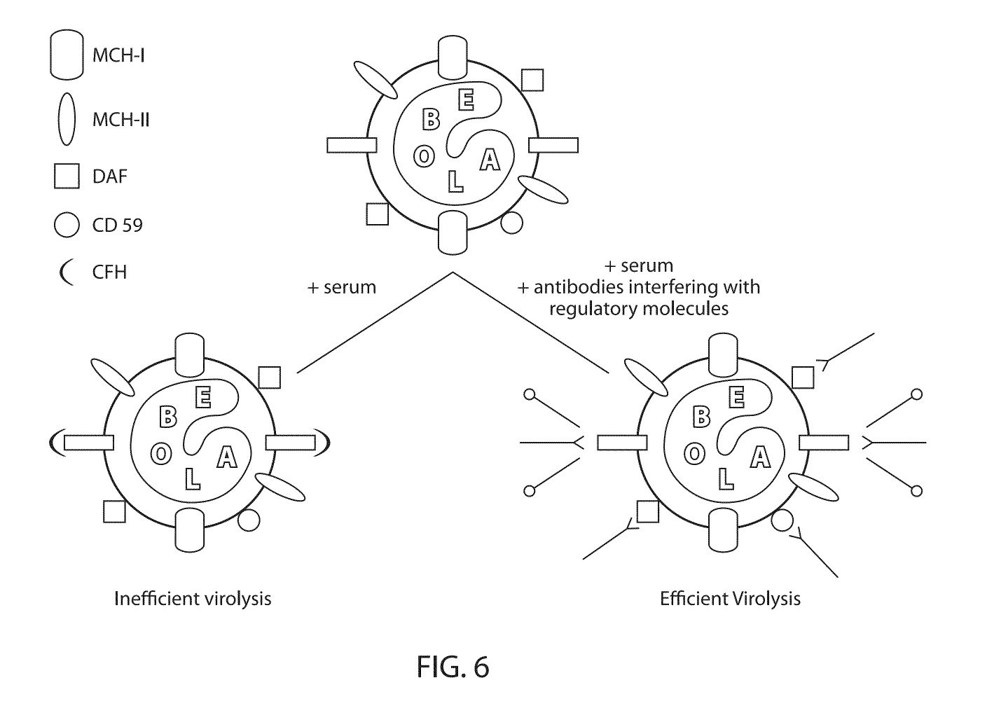

2. For Generating Host specific vaccine responses: In asymptomatic patients: The proposed dose is less than 50 microgram per kg body weight. At this dosage, circulating Antibodies generates Host specific vaccine responses. Under normal circumstances the circulating antibodies don't sense the presence of virus due to the presence of Factor H coat on Ebola cell surface. Antibodies in fact enhance infectivity of virus (Takada, Ayato. et al. Journal of Virology. Antibody-Dependent Enhancement of Ebola Virus Infection, 2003, 77 (13):7539, DOI: 10. 1128/JVI. 77.13.7539-7544.2003 (24). The removal of Factor H by NSPS or Factor H Ab, generates prompt efficient virolysis as per FIG. 6. FIG. 6 is a diagram showing the generation of Host specific immune responses. Asymptomatic carriers, patients and animal harboring viruses may have circulating antibodies. Therefore, NSPs can be used in micro quantity to sterilize and decontaminate sources of infection.

3. Preventing host immune adverse effects: As shown in FIG. 8. NSPS three dimensionally target Factor D and Factor H. Factor D targeting reduces or inhibits host inflammatory responses while targeting Factor H on viral surfaces, inhibit immune evasion mechanism prompting host and its circulating Antibodies to generate cytotoxic immune responses.

4. Strategy for Decontamination of Infectious waste and infectious environment. NSPS like Ebola and Factor H has strong electrostatic properties to bind complement regulatory and activating proteins. Factor H is a 155 KD Protein that is unlike other immune regulatory proteins are present in circulation as well as in infectious liquid waste. Therefore its binding with Ebola can readily be neutralized by spraying NSPS over liquid waste of patients to render it noninfectious. Similar strategy can be extended to decontaminate clothing, patient environment as well as work place environments in both laboratories and hospital settings.

5. Strategy for Sterilization of Asymptomatic carriers: Asymptomatic carriers such as select animals and asymptomatic patients harbor both the virus and circulating antibodies. The use of NSPS in microgram quantities by removing Factor H will prime host specific immune responses to destroy viruses and sterilize asymptomatic carriers and animal's viral reserviors.

6. Strategy of generating vaccine responses: NSPS can be readily cross linked with any antibodies or antigen of specific pathogens and can be used to generate vaccine responses either through intravenous injection (NSPS-AB Conjugate) or Subcutaneously or nasal route for vaccine responses (NSPS-Ag conjugate).

Having described preferred embodiments for (which are intended to be illustrative and not limiting), it is noted that persons can make modifications and variations skilled in the art in light of the above teachings. In practicing the formulation methods, alternate or additional steps may be included that do not alter the purpose of the invention. The use of equivalent materials other than those specified is intended to be included within the scope of the invention. It is therefore to be understood that changes may be made in the particular embodiments of the invention disclosed which are within the scope and spirit of the invention as outlined by the appended claims. Having thus described the invention with the details and particularity required by the patent laws, what is claimed and desired protected by Letters Patent is set forth in the appended claims.

1. A method for treating deadly pathogens having an immune regulatory molecule comprising:
providing a nano-engineered formulation of sodium polystyrene sulfonate (NSPS) having particle size less than 100 nm;
administering a pharmaceutically effective dose between 5 and 100 mg of the NSPS suspended in one of sterile water and normal saline to form a mixture to a patient infected with the pathogen; and
targeting the immune regulatory molecule with the NSPS for inhibiting serine protease activation.

2-3. (canceled)

4. The method of claim 1, wherein immune pathogenesis of deadly pathogens can be treated expediently and globally with NSPS to mitigate the threat of bioterrorism and pandemics.

5. The method of claim 4, wherein said providing step includes one of providing NSPS and providing milligram dosages of NSPS; and
wherein said targeting step includes targeting one or more of the group consisting of Serine proteases of coagulation cascade, classical complement system cascade, Lectin based complement cascade, Alternate complement based cascade and combinations thereof.

6. The method of claim 5, wherein said targeting step includes using NSPS to down regulate host inflammatory, bleeding and cytokine inflammatory response.

7-8. (canceled)

9. The method of claim 1, wherein the deadly pathogen is Ebola.

10. The method of claim 1, wherein said administering step comprises:
administering an oral single daily dose of 5 mg NSPS per kg of the patients body weight.

11. The method of claim 1, wherein said administering step comprises:
administering by injection a 5 mg NSPS per kg of the patients body weight suspended in one of sterile water or normal saline.

12. The method of claim 1, wherein said administering step comprises:
administering by nasal spray 10-100 mg NSPS suspended in normal saline.

13. The method of claim 1, wherein said providing step includes centrifuging patient matched blood and collecting cells; combining 10-100 mg NSPS with the collected cells and incubating; and adding sterile solution to form a mixture; and
wherein said administering step includes administering the mixture intravenously; and
wherein said targeting step includes linking the NSPS to one or more of RBC, platelets, monocytes and white blood cells for removing interfering immune regulatory molecules and priming cytotoxic immune responses.

14. The method of claim 1, wherein said providing step includes centrifuging patient matched blood and collecting cells; combining 10-100 mg NSPS with the collected cells and incubating; and adding sterile solution to form a mixture; and
wherein said administering step includes administering the mixture intravenously; and
wherein said targeting step includes linking the NSPS to suitable monoclonal antibodies for generating cytotoxic immune responses.

15. The method of claim 1, wherein said providing step includes centrifuging patient matched blood and collecting cells; combining 10-100 mg NSPS with the collected cells and incubating; and adding sterile solution to form a mixture; and
wherein said administering step includes administering the mixture subcutaneously; and
wherein said targeting step includes linking the NSPS with Ebola antigen for generating vaccine responses in healthy patients.

16-17. (canceled)
